(19) United States
(12) Patent Application Publication
(10) Pub. No.: US 2005/0288865 A1
(43) Pub. Date: Dec. 29, 2005

(54) Peptide and protein identification method

(75) Inventors: Ron D. Appel, Meinier (CH); Robin Gras, Annemasse (FR); Patricia Hernandez, Versoix (CH)

Correspondence Address: DECHERT LLP, P.O. BOX 10004, PALO ALTO, CA, 94303, US

(73) Assignee: Institut Suisse de Bioinformatique, Geneva (CH)

(21) Appl. No.: 11/030,301

(22) Filed: Jan. 7, 2005

Related U.S. Application Data
(63) Continuation of application No. PCT/IB02/02731, filed on Jul. 10, 2002.

(51) Int. Cl. C12Q 1/68; G06F 19/00

(52) U.S. Cl. 702/19

(57) ABSTRACT

A method for identifying peptides and proteins, starting from corresponding tandem spectrometry data. A structured representation is matched with a biological sequence database, and the best peptide match or matches within the database is determined. MS/MS data is interpreted and structured to allow full exploitation of the information contained in the data during matching of the structured data with a biological sequence database.

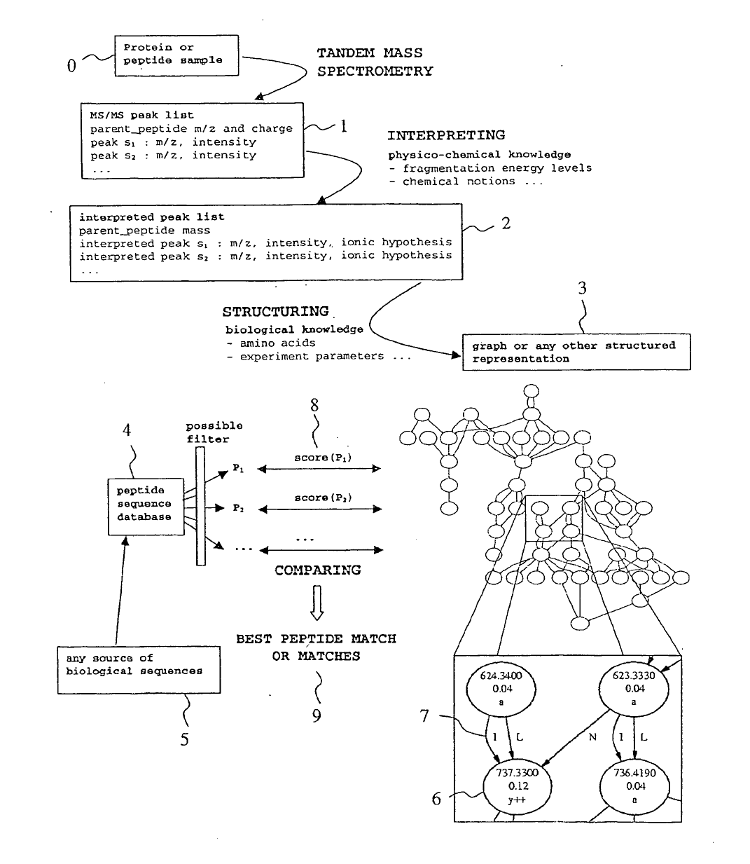

### BACKGROUND OF THE INVENTION

1. Field of the Invention

This invention relates to the field of proteomics and particularly to methods and systems for identifying peptides and proteins starting from tandem spectrometry data (MS/MS data) obtained experimentally. More specifically, the method comprises interpreting and structuring MS/MS data in a way allowing full exploitation of the information contained in it during matching of the structured data with biological sequence database.

The information provided herein has previously been published as International Application WO 2004/008371, which is hereby incorporated by reference in its entirety.

The following references are either cited in the text or relevant to the prior art:
Bafna V. and Edwards N. (2001). SCOPE: a probabilistic model for scoring tandem mass spectra against a peptide database. Bioinformatics Suppl. 1, 13-21.
Bairoch, A. and Apweiler, R. (2000). The SWISS-PROT protein sequence database and its supplement TrEMBL in 2000. Nucleic Acids Res. 28, 45-48.
Barker, W. C., Garavelli, J. S., Huang, H., McGarvey, P. B., Orcutt, B. C., Srinivasarao, G. Y., Xiao, C., Yeh, L. S., Ledley, R. S., Janda, J. F., Pfeiffer, F., Mewes, H. W., Tsugita, A., and Wu, C. (2000). The protein information resource (PIR). Nucleic Acids Res. 28, 41-44.
Bartels C. (1990). Fast algorithm for peptide sequencing by mass spectrometry. Biomed. Environ. Mass. Spectrom. 19, 363-368.
Benson, D. A., Karsch-Mizrachi, I., Lipman, D. J., Ostell, J., Rapp, B. A., and Wheeler, D. L. (2002). GenBank. Nucleic Acids Res. 30, 17-20.
Bonabeau E., Dorigo M., and Theraulaz G. (1999). Swarm Intelligence. From Natural to Artificial Systems. Oxford University Press).
Chen, T., Kao, M. Y., Tepel, M., Rush, J., and Church, G. M. (2001). A dynamic programming approach to de novo peptide sequencing via tandem mass spectrometry. J. Comput. Biol. 8, 325-337.
Clauser K. R., Hall S. C., Smith D. M., Webb J. W., Andrews L. E., Tran H. M., Epstein L. B., and Burlingame A. L. (1995). Rapid mass spectrometric peptide sequencing and mass matching for characterization of human melanoma proteins isolated by two-dimensional PAGE. Proc. Natl. Acad. Sci. USA 92(11), 5072-5076.
Dancik, V., Addona, T. A., Clauser, K. R., Vath, J. E., and Pevzner, P. A. (1999). De novo peptide sequencing via tandem mass spectrometry. J. Comput. Biol. 6, 327-342.
Dorigo, M. and Di Caro, G. (1999). The Ant Colony Optimization Meta-Heuristic. In New Ideas in Optimization, D.M.G.F.E. Corne D., ed.
Edman, P. (1970). Sequence determination. Mol. Biol. Biochem. Biophys. 8, 211-255.
Eng J. K., McCormack, A. L., and Yates, I. J. R. (1994). An approach to correlate tandem mass spectral data of peptides with amino acid sequences in a protein database. J. Am. Soc. Mass Spectrom. 5, 976-989.
Fenyo, D., Qin, J., and Chait, B. T. (1998). Protein identification using mass spectrometric information. Electrophoresis 19, 998-1005.
Fernandez-de-Cossio, J., Gonzalez, J., and Besada, V. (1995). A computer program to aid the sequencing of peptides in collision-activated decomposition experiments. Comput. Appl. Biosci. 11, 427-434.
Fernandez-de-Cossio, J., Gonzalez, J., Betancourt, L., Besada, V., Padron, G., Shimonishi, Y., and Takao, T. (1998). Automated interpretation of high-energy collision-induced dissociation spectra of singly protonated peptides by “SeqMS”, a software aid for de novo sequencing by tandem mass spectrometry. Rapid Commun. Mass Spectrom. 12, 1867-1878.
Fernandez-de-Cossio, J., Gonzalez, J., Satomi, Y., Shima, T., Okumura, N., Besada, V., Betancourt, L., Padron, G., Shimonishi, Y., and Takao, T. (2000). Automated interpretation of low-energy collision-induced dissociation spectra by SeqMS, a software aid for de novo sequencing by tandem mass spectrometry. Electrophoresis 21, 1694-1699.
Gatlin, C. L., Eng, J. K., Cross, S. T., Detter, J. C., and Yates, J. R., II (2000). Automated identification of amino acid sequence variations in proteins by HPLC/microspray tandem mass spectrometry. Anal. Chem. 72, 757-763.
Gonnet G. H. A tutorial Introduction to Computational Biochemistry Using Darwin. 1992. E. T. H. Zurich, Switzerland. Ref Type: Report
Gras, R., Muller, M., Gasteiger, E., Gay, S., Binz, P. A., Bienvenut, W., Hoogland, C., Sanchez, J. C., Bairoch, A., Hochstrasser, D. F., and Appel, R. D. (1999). Improving protein identification from peptide mass fingerprinting through a parameterized multi-level scoring algorithm and an optimized peak detection. Electrophoresis 20, 3535-3550.
Gras R., Gasteiger E., Chopard B., Müller M., and Appel R. D. New learning method to improving protein identification from peptide mass fingerprinting. 2000. 4^{th} Siena 2D electrophoresis meeting. Ref Type: Conference Proceeding
Gras R. and Muller M. (2001). Computational aspects of protein identification by mass spectrometry. Current Opinion in Molecular Therapeutics 3, 526-532.
Hines W. M., Falick A. M., Burlingame A. L., and Gibson B. W. (1992). Pattern-based algorithm for peptide sequencing from tandem mass spectra of peptides. J. American Society for Mass Spectrometry 3, 326-336.
Ishikawa, K. and Niwa, Y. (1986). Computer-aided peptide sequencing by fast atom bombardment mass spectrometry. Biomed. Environ. Mass Spectrom 13, 373-380.
Johnson, R. S. and Biemann, K. (1989). Computer program (SEQPEP) to aid in the interpretation of high-energy collision tandem mass spectra of peptides. Biomed. Environ. Mass Spectrom 18, 945-957.
Johnson, R. S. and Taylor, J. A. (2000). Searching sequence databases via de novo peptide sequencing by tandem mass spectrometry. Methods Mol. Biol. 146, 41-61.
Kennedy J. and Eberhart R. C. (2001). Swarm Intelligence. Morgan Kaufmann).
Mann, M., Hojrup, P., and Roepstorff, P. (1993). Use of mass spectrometric molecular weight information to identify proteins in sequence databases. Biol. Mass Spectrom 22, 338-345.
Mann, M. and Wilm, M. (1994). Error-tolerant identification of peptides in sequence databases by peptide sequence tags. Anal. Chem. 66, 4390-4399.
Pappin D. D. J., Hojrup P., and Bleasby A. J. (1993). Rapid identification of proteins by peptide-mass finger printing. Curr. Biol. 3, 327-332.
Perkins D. N., Pappin D. D. J., Creasy D. M., and Cottrell J. S. (1999). Probability-based protein identification by searching sequence databases using mass spectrometry data. Electrophoresis 20, 3551-3567.
Pevzner, P. A., Dancik, V., and Tang, C. L. (2000). Mutation-tolerant protein identification by mass spectrometry. J. Comput. Biol. 7, 777-787.
Pevzner, P. A., Mulyukov, Z., Dancik, V., and Tang, C. L. (2001). Efficiency of database search for identification of mutated and modified proteins via mass spectrometry. Genome Res. 11, 290-299.
Sakurai T., Matsuo T., Matsuda H., and Katakuse I. (1984). Paas 3: A computer program to determine probable sequence of peptides from mass spectrometric data. Biomed. Mass Spectrom. 11(8), 396-399.
Siegel, M. M. and Bauman, N. (1988). An efficient algorithm for sequencing peptides using fast atom bombardment mass spectral data. Biomed. Environ. Mass Spectrom. 15, 333-343.
Stoesser, G., Baker, W., van den, B. A., Camon, E., Garcia-Pastor, M., Kanz, C., Kulikova, T., Leinonen, R., Lin, Q., Lombard, V., Lopez, R., Redaschi, N., Stoehr, P., Tuli, M. A., Tzouvara, K., and Vaughan, R. (2002). The EMBL Nucleotide Sequence Database. Nucleic Acids Res. 30, 21-26.
Tateno, Y., Imanishi, T., Miyazaki, S., Fukami-Kobayashi, K., Saitou, N., Sugawara, H., and Gojobori, T. (2002). DNA Data Bank of Japan (DDBJ) for genome scale research in life science. Nucleic Acids Res. 30, 27-30.
Taylor, J. A. and Johnson, R. S. (1997). Sequence database searches via de novo peptide sequencing by tandem mass spectrometry. Rapid Commun. Mass Spectrom. 11, 1067-1075.
Taylor, J. A. and Johnson, R. S. (2001). Implementation and uses of automated de novo peptide sequencing by tandem mass spectrometry. Anal. Chem. 73, 2594-2604.
Wilkins M. R., Gasteiger E., Bairoch A., Sanchez J. C., Williams K. L., Appel R. D., and Hochstrasser D. F. (1999a). Protein identification and analysis tools in ExPASy server. Methods Mol Biol 112, 531-552.
Wilkins M. R., Gasteiger E., Wheeler C. H., Lindskog I., Sanchez J. C., Bairoch A., Appel R. D., Dunn M. J., and Hochstrasser D. F. (1999b). Multiple parameter cross-species protein identification using Multident—a world-wide web accessible tool. Electrophoresis 19, 3199-3206.
Yates, I. J. R., Eng J. K., and McCormak A. L. (1995). Mining genomes: correlating tandem mass spectra of modified and unmodified peptides to sequences in nucleotide databases. Anal. Chem. 67(18), 3202-3210.
Yates III. J. R., Eng J. K., Clauser K., and Burlingame A. L. (1996). Search of Sequence Databases with Uninterpreted High-Energy Collision-Induced Dissociation Spectra of Peptides. J. American Society for Mass Spectrometry 7, 1089-1098.
Zhang, W. and Chait, B. T. (2000). ProFound: an expert system for protein identification using mass spectrometric peptide mapping information. Anal. chem. 72, 2482-2489.

2. Description of the Prior Art

Proteomics is the study of the proteins resulting from the expression of the genes contained in genomes. Due to important variations of protein expression between cells having the same genome, there are many proteomes for each corresponding genome. As a result, huge amounts of information are involved, and the study of proteome is even more complex than the study of the genome.

A typical goal of proteomics is to identify the protein expression in a given tissue or cell under given conditions. An additional goal of proteomics is to compare the protein expression in the same tissue, cell or physiological fluid under varying conditions (for example disease vs. control), and identify the proteins that are differently expressed.

In recent years, proteomics research has gained importance due to increasingly powerful techniques in protein purification/separation, mass spectrometry and identification techniques, as well as the development of extensive protein and nucleic databases from various organisms.

A traditional method for analyzing proteomes involves separation by 1-D and 2-D polyacrylamide-gel electrophoresis. The 1-D gel method is generally used to achieve a crude separation of cell lysates where the most abundant proteins can be separated and detected. 2-D gel electrophoresis is a more powerful method capable of separating out hundreds of protein spots, where the spot pattern is characteristic of protein expression. Typical separation criteria by gel electrophoresis include electrical charge (isoelectric point—pI) and molecular weight. Gel electrophoresis methods (1-D and 2-D) have nevertheless certain fundamental limitations for screening and identification of proteins. Notably, gel electrophoresis separations are slow and have a limited resolution (i.e. can only distinguish between a limited number of proteins (spots)). In recent years, automation has allowed to manage larger quantities of data resulting from 2-D gel electrophoresis, as exemplified by U.S. Pat. No. 5,993,627, U.S. Pat. No. 6,277,259, and WO 00/55636.

Higher resolution can be attained by other chromatography separation methods such as capillary electrophoresis, gas chromatography, micro-channel networks, liquid chromatography and high-pressure liquid chromatography (HPLC), used in complement to gel electrophoresis or alone. These methods allow the separation of greater numbers of proteins, even in hard conditions (low sample quantities, small molecular weight, highly basic or hydrophobic proteins . . . ). Separation criteria include electrical charge and molecular weight as in gel electrophoresis, as well as hydrophobicity and other physico-chemical criteria.

After separation, the proteins must be identified, by sequencing or other means. Determining the sequence of amino acid residues in a protein was traditionally accomplished by means of N-terminal Edman degradation (Edman, 1970). Edman sequencing unfortunately requires important quantities of a protein (in the order of 10-100 pmols), which exceed the quantities obtained from most current separation techniques. In practice, Edman sequencing is possible only after 1-D or 2-D gel electrophoresis, and then only for the most abundant protein species found.

Today, most large-scale protein identification procedures use mass spectrometry (MS) data as a starting point rather than Edman degradation. Mass spectrometry accurately determines the molecular mass of the analyzed protein. Additional information can be obtained by cleavage of the protein into smaller peptides before performing the mass spectrometry. Cleavage of proteins is usually done by enzymatic means, most commonly by trypsin which cleaves specifically the C-terminal side of arginine or lysine.

There are several identification methods from mass spectrometry data (Gras and Muller, 2001). The most widely used method consists in measuring masses of peptides resulting from the digestion process by mass spectrometry. The resulting MS spectrum represents a peptide mass fingerprint (PMF), which is characteristic for each protein. Identification by peptide mass fingerprint requires a pre-existing protein database, either directly produced or derived from a nucleic database. Identification is done by comparing the experimental masses/spectra obtained by MS (PMF) and the theoretical masses/spectra of virtually digested protein sequences present in the database. The shared masses between the experimental and theoretical spectra are used in a more or less elaborated scoring function to identify the protein. Some tools only count the number of matches, such as PepSea (Mann et al., 1993), PeptideSearch (Mann and Wilm, 1994), PeptIdent/MultIdent (Wilkins et al., 1999a; Wilkins et al., 1999b), while others use a probabilistic and/or statistic approach, such as MassSearch (Gonnet, 1992), MOWSE (Pappin et al., 1993), MS-Fit (Clauser et al., 1995), Mascot (Perkins et al., 1999), ProFound (Zhang and Chait, 2000). Finally, the algorithm developed by Gras, Smartldent (Gras et al., 1999; Gras et al., 2000), uses a machine learning approach.

Unfortunately, the PMF method may not always succeed in giving a reliable identification, for example when the concentration of the protein of interest is low, when only a few peptides are found after the digestion process or when the protein of interest is insufficiently purified. In addition, post-translational modifications (PTMs) or polymorphisms may modify the peptide masses and impair proper matching. Finally, it is possible that the protein of interest is simply not present in the protein database, and therefore cannot be matched.

In cases where identification is uncertain, one can use tandem mass spectrometry (MS/MS). MS/MS spectra are obtained after selection of a peptide coming from the digestion process of the protein of interest, subsequent fragmentation of said peptide (for example, by collision with a rare gas), and measurement of the produced fragment masses. Ideally, fragmentation occurs between every amino acid of the peptide, and the masses of two adjacent ionic peaks differ by the mass of one amino acid. In addition to a PMF similar to the one obtained from MS identification, MS/MS data provide information concerning the peptide sequence and allow a more detailed interpretation level than MS spectra alone.

Exploiting the information contained in MS/MS spectra is difficult due to various factors. Notably, the fragmentation process is hardly foreseeable and depends, among other things, on the amount of energy used by the mass spectrometer, on the number and the repartition of the charges carried by the ionic fragment, on its sequence, etc.

Two main identification strategies have been devised to exploit MS/MS data: de novo sequencing followed by sequence matching, and direct spectrum matching with theoretical spectra from an existing database.

De novo sequencing consists in deriving a peptide sequence from its MS/MS spectrum without use of any information extracted from a pre-existing protein or nucleic database. To do so, de novo sequencing uses not only the mass values represented by peaks in the mass spectra, but also their position respective to each other. Early methods required generating all possible sequences whose masses are similar to the spectrum's parent mass and all the corresponding virtual spectra, PAAS3 (Sakurai et al., 1984). The experimental spectrum was then compared and matched with the virtual spectra. This approach was rapidly abandoned due to the combinatorial explosion it implies. Another strategy was to make successive possible extension of sequences (Ishikawa and Niwa, 1986). The sequences are built by successive extension with one or more amino acids. For each iteration, the sub-sequences and the corresponding virtual spectra are compared with the experimental spectrum, and the most divergent sequences are eliminated. Still another, more sophisticated strategy uses the information lying in the succession of the peaks to make the sequence extensions (Siegel and Bauman, 1988), SEQPEP (Johnson and Biemann, 1989). In this approach, the peptide sequence is built step by step, from the masses differences of “neighbor” peaks in the spectrum. This method can be viewed as the precursor of methods based on graph representation (Bartels, 1990), (Hines et al., 1992), SeqMS (Fernandez-de-Cossio et al., 1995; Fernandez-de-Cossio et al., 1998; Femandez-de-Cossio et al., 2000), Lutefisk97 (Taylor and Johnson, 1997; Johnson and Taylor, 2000; Taylor and Johnson, 2001), SHERENGA (Dancik et al., 1999), (Chen et al., 2001). The vertices in the graph are built from the peaks of the spectrum and represent masses of potential fragments. Physico-chemical properties are taken into account to associate a score to each vertex. Whenever two vertices differ by the mass of one or several amino acid, they are connected by an arc. Therefore, each path in the graph represents a possible sequence that can be built from the spectrum. Special algorithms then search the graph for the best paths (i.e. having the highest score built from the vertices score belonging to the path), allowing determining the most probable sequence or sequences corresponding to the experimental spectrum. Accordingly, de novo sequencing results in one or a limited number of possible amino acid sequence, obtained without any recourse to a protein or nucleic database.

For identification purposes, the sequence(s) (partial or complete) obtained de novo are then used to scan a protein database with a standard alignment software. De novo sequencing is a fairly complex task which requires both good quality spectra and manual verification by a mass spectrometry expert. Accordingly, this approach is not adapted to the huge amounts of data generated by high-throughput settings available today.

The alternative to de novo sequencing is to match the experimental peptide spectra obtained from MS/MS with theoretical spectra derived from pre-existing protein databases. Unlike de novo sequencing, most MS/MS spectra matching tools use only the mass values in the MS/MS spectra—to the exclusion of their respective positions. The method most used today for MS/MS identification is the shared peak count (SPC). The ionic masses of the MS/MS spectrum represent an “ion mass fingerprint”, by analogy with the “peptide mass fingerprint”. The experimental MS/MS spectrum is compared with theoretical ion mass fingerprints of virtually digested and fragmented proteins in the database. Their similarity is determined by a combination of independent scores of correlations between the experimental and theoretical common masses.

Various SPC algorithms have been developed. All are based on a probabilistic score depending on the mass errors and differ mainly by their scoring function, which can be more or less sophisticated. MSTag, PepFrag (Fenyo et al., 1998), and MASCOT (Perkins et al., 1999) are examples. One algorithm—SCOPE (Bafna and Edwards, 2001)—uses both a complex probabilistic model and a dynamic programming method. Another algorithm, SEQTJEST (Eng et al., 1994; Yates et al., 1995; Yates et al., 1996; Gatlin et al., 2000), uses two filtering levels: SPC followed by cross-correlation by means of fast Fourier transformation. Concerning modifications, any mutation or PTM of the source protein is susceptible to drastically modify the MS/MS spectra in comparison to the unmodified protein in the reference database: modified fragment masses are shifted by a delta corresponding to the mass difference brought by the modification/mutation. As a result, a source modified peptide might not find any corresponding match in the reference protein database. SPC methods generally include in the database all modified/mutated peptides that they want to consider, which requires prior knowledge of the mass difference associated with the modifications/mutations taken into account. Accordingly, modifications whose mass difference with the unmodified peptide is unpredictable (such as glycosylations) cannot be taken into account by SPC methods. In addition, including all possible modifications/mutations of the peptides in the database is unrealistic due to the combinatorial explosion it implies. As a result, SPC methods usually take into account only a few very common modifications occurring on specific amino acids, such as methionine oxidation or cysteine carbamidomethylation.

In addition to the combinatorial problem, SPC algorithms have two other limitations. First, they consider the peaks independently of each other, thereby losing some important information contained in MS/MS spectra. Second, SPC algorithms need to allow a large error tolerance when used with badly calibrated spectra. As a result, the high intrinsic accuracy of current mass spectrometers is basically lost.

Two non-SPC methods have been described: spectral convolution and spectral alignment, with PEDANTA (Pevzner et al., 2000; Pevzner et al., 2001) their corresponding tool, which are claimed to be very efficient in dealing with modifications/mutations, including unpredictable modifications. Indeed, they have a major advantage over SPC methods, because they use logical constraints imposed by the spectrum peak composition to limit the number of considered modifications/mutations. One obvious trade-off of these approaches is that one must parse the whole peptide database without using the parent mass as filtering. In addition, the combinatorial problem grows with the number of contemplated mass shifts. Accordingly, the number of modifications/mutations considered must be kept sufficiently low in order to allow identifications that are sufficiently discriminating.

### SUMMARY OF THE INVENTION

According to the present invention, tandem spectrometry data (MS/MS data) obtained experimentally from peptide and/or protein-containing samples is interpreted and structured in a way allowing full exploitation of the information contained in it during matching of the structured data with biological sequence database.

### BRIEF DESCRIPTION OF THE DRAWING

The figure is a flow chart showing the general pathway of the method for identifying peptides or proteins from MS/MS data according to an embodiment of the present invention.

### DESCRIPTION OF THE INVENTION

The present invention concerns a peptide and protein identification method of tandem spectrometry, such as, for example, ESI/MALDI Q-TOF MS, ESI/MALDI Ion-Trap MS, ESI triple quadrupole MS or MALDI TOF-TOF MS. Instead of directly comparing the experimental MS/MS spectrum with theoretical sequences from the database as in SPC, the method of the present invention compares an interpreted and structured view of the experimental MS/MS spectrum with theoretical sequences.

In the method of the invention and referring to the figure, one first performs tandem spectrometry on a sample 0, containing one or more protein or peptide. The MS/MS spectrum is then translated into a peak list 1, listing discrete mass peaks. This step can be performed by standard mass spectrometry equipment. The resulting peak list 1 is then interpreted into a list of possible mass explanations (interpreted peak list 2) taking into account physico-chemical knowledge, notably concerning the mass spectrometer, fragmentation energy levels and chemical notions (ion type, charge number, etc.). The interpreted peak list 2 is then transformed into a structured representation 3, taking into account biological knowledge—notably amino acid properties, and preserving at least the following information:
Mass/charge ratio of the peaks
Mass/charge ratio of the parent peptide
Charge of the parent peptide
Intensity of the peaks

Identification of the peptide is performed by matching said structured representation with a biological sequence database. Said database 4 is built from any source of biological sequences 5 such as a nucleic database translated into a protein or peptide database, or any subset of such databases. A number of sequence libraries can be used, including for example GenBank (Benson et al., 2002), EMBL (Stoesser et al., 2002), DDBJ (Tateno et al., 2002), SWISSPROT (Bairoch and Apweiler, 2000), and PIR (Barker et al., 2000). The matching with the biological sequence database is performed prior to any reduction of the structured representation 3 into one or a limited number of amino acid sequences, in contrast to de novo sequencing. The matching process leads to a similarity score 8 for each peptide sequence. This score is then used to determine the best peptide match or matches 9.

The present invention also provides a protein identification method comprising the steps of the peptide identification method just described, and comprising a further step consisting in using the peptide matching information for identification of the corresponding protein or proteins in a protein database.

In a preferred embodiment of the invention, the structured representation matched with the database is a graph 3 wherein vertices 6 of the graph 3 represent “ideal” fragments, built from MS/MS peaks (in the interpreted peak list 2) under an ionic hypothesis. Each vertex 6 representing a fragment indicates among others the molecular mass value of said fragment, the specific ionic hypothesis (ion type) for this fragment, and is assigned a score value expressing the credibility level for the vertex. Two vertices 6 are connected by an edge 7 whenever their mass difference is equivalent to the mass value of one or more amino acids, depending on the combinatorial level chosen. Letters representing these specific amino acids are attached to the edge 7. Accordingly, the graph 3 represents all amino acid tags and complete sequences that can possibly be built from the MS/MS spectrum. Identification of the best peptide match or matches 9 is performed using the similarity scores 8 obtained by comparing theoretical peptides from the peptide sequence database 4 and the graph 3.

The method of the present invention compares the structured representation (or graph) 3 with theoretical peptides from a peptide sequence database 4. In contrast to identification by de novo sequencing followed by sequence matching—that uses database information only after reduction of the graph to one or several sequences, the present invention directly uses database information to direct the comparison with the structured representation or graph. The goal is to find sections (sets of consecutive edges 7) of the structured representation or graph 3 which best explain the peptide. Although a section can be viewed as a classical tag encompassing sequence information, it is more than that as it contains additional information used in the comparison process.

In the present invention, the structured representation in general, and the graph structure in particular, have significant advantages over existing methods. This approach first eliminates the calibration issue during the comparison process. As already mentioned, peak masses in MS/MS spectra can be shifted of a significant value in spite of the high intrinsic accuracy of the spectrometer. As a result, existing identification methods based on SPC must allow for a high tolerance error when comparing peak masses and theoretical fragment masses, which leads to a significant increase of the noise level, hence of the number of false positives. The method of the present invention compares, differences of peak masses with differences of theoretical masses. Because differences of adjacent masses are weakly influenced by calibration errors, the method of the present invention allows to fully taking advantage of the spectrometer accuracy. Another advantage of the structured representation is that it allows to take into account not only the number of peak matches (as in SPC), but also the number of successive matches susceptible to explain the sequence.

In a preferred embodiment of the invention, the matching of the structured representation with sequences in the database is performed by parsing the structured representation or the graph according to each database sequence, each parsing leading to a score correlating each database sequence to the structured representation or graph.

This approach allows notably comparing the structured representation with any sub-sequences of the peptide sequence database, each parsing leading to a score correlating the sub-sequence with a section of the structured representation or graph. In case of incomplete spectral information, non-linked relevant sets of successive edges (sections) can be combined together to form a same peptide sequence. In case of modified source peptides, this approach also allows to combine non-linked relevant sets of successive edges (sections) according to a modification hypothesis.

Representations under a graph structure allow to keep all the original information, as well as to consider information coming from many different sources during the comparison process. The graph includes two information types: first, local information, which are used for the path building in order to favor most pertinent edges and which are stored in variables associated with vertices and edges (as the vertices mass, intensity, score or the edge amino acid), and second, global information, which describe path pertinence related to the current peptide or to any subsequence belonging to it, and possibly stored in weights associated with edges. Local and global parameters must be weighted and combined in a way maximizing the performance of the identification algorithm, and allowing sufficient discrimination between the peptide ranked first and the other candidates. Using a set of identified spectra from a known mass spectrometer, it is possible to optimize the weights with genetic algorithms (Gras et al., 2000; Gras et al., 1999).

In another embodiment of the invention, said parsing is performed through the use of a Swarm Intelligence-type algorithm (Kennedy and Eberhart, 2001; Bonabeau et al., 1999). Swarm intelligence is a form of distributed artificial intelligence: self-organization of unsophisticated units—agents, evolving and interacting within a given environment and able to manage direct and/or indirect communication, results in the emergence of an intelligent collective behavior.

In still another embodiment of the invention, the Swarm Intelligence-type algorithm is an algorithm called “Ant Colony Optimization” (ACO) (Dorigo and Di Caro, 1999). ACO algorithms are defined as multi-agent systems inspired from real ant colony behavior. The principle of ACO is to explore, iteratively and simultaneously, different solutions of a given problem by an ant-agent population. The emergent collective behavior is guided by indirect communication between the ants, mediated by environmental modifications (stigmergy). Ants modify their environment by depositing given amounts of pheromone, which are locally accessible and affects the behavior of the other ants. In this embodiment, an ACO algorithm inspired from the “trail-laying/trailfollowing” foraging behavior of ants is used to score the matching of current peptide of the database with the structured representation. Since ants can find the shortest path connecting the colony to the food source, it is possible to exploit the rules governing the foraging process and use them to find good scoring paths in the graph. Each “ant” obtains a score depending on the quality of the found solution. The use of virtual pheromone allows good solutions to be memorized and act as a positive feedback (intensification of the search). In order to avoid premature convergence, a certain amount of pheromone also evaporates at each iteration (negative feedback, diversification of the search). The modified ACO used to parse the graph first sets the pheromone quantity of each edge to a tiny value. Then, the ants parse the graph iteratively. At each iteration, the ants move on the graph from one vertex to the other, using existing edges or, if allowed, jumping from one vertex to the other until a stop criterion is reached (for example, when arrived on a vertex having no successor). The choice of the next edge results from a probabilistic computation, taking into account both local parameters (i.e. the score of the successor vertex) and the global learning already done (i.e. the amount of pheromone on the successor edge). At the end of each iteration, some pheromone is automatically removed from each edge (evaporation), while some pheromone is added on each edge parsed by an ant (the exact amount being dependent on the ant's score). As a result, the algorithm allows gradual convergence toward one or several good scoring sections, which can be further correlated in order to maximally cover the theoretical candidate peptide, ultimately leading after analysis of all peptides to a ranked list of candidate peptides.

The ACO algorithm has several advantages. For example, the stochastic nature of the ant motion allows parsing any path in the graph. All possible mutations compatible with the MS/MS spectrum are implicitly represented in the graph, and possible modifications can be contemplated by allowing the ants to jump from one vertex to another, unconnected one. Like spectral alignment methods, the present invention uses the spectrum logical constraints to limit the combination number of possible modifications. In addition, it drastically restricts this number by allowing only directed jumps joining relevant sections of the representation or graph. Thus, only modifications enhancing the global correspondence between the sequence and the spectrum are considered. It is also possible to restrict the vertices allowed for an ant, depending on the vertices already parsed by this ant. This allows accepting, for example, only one missed-cleavage: an ant having used an edge corresponding to a lysine could avoid to further incorporate a second lysine.

An additional advantage of the present invention is that switching from it to a more traditional de novo sequencing mode is straightforward, by simply letting aside, the information coming from the database.

The invention also provides a system comprising a computer linked to one or more mass spectrometers and one or more biological sequence databases, said computer comprising a program for performing the steps of the methods described herein.

The invention also provides a computer-readable medium comprising instructions for causing a computer linked to one or several mass spectrometers and to one or more biological sequence databases to perform the steps of the methods described herein.

### DETAILED DESCRIPTION OF A PREFERRED EMBODIMENT

The following paragraphs provide a detailed description of MS/MS data treatment and identification according to a preferred embodiment of the invention, combining a graph representation and an ACO algorithm and called Popitam (Peptide Or Protein Identification from Tandem Mass Spectrometry).

I. Peak Interpretation

Let us define sₑₓₚ={s₁,s₂, . . . ,s_{|S}_{ₑₓₚ}_{|}}, the experimental MS/MS peak list to identify, and a set of ionic hypothesis Δ={η₁,η₂, . . . ,η_{|Δ|}}. An ionic hypothesis can be seen as a possible interpretation of a peak. Each ηᵢ has four attributes, which are presumptions concerning the ionic fragment sⱼ measured by the spectrometer: an offset value o(ηₖ), i.e. the mass difference between the ionic fragments and the corresponding b-ion type fragment (for comprehension purpose, we will call such fragments b-fragments, and their corresponding masses b-masses), a terminus side t(ηₖ) (N-term or C-term), a number of charges c(ηₖ), and an approximated occurrence probability p(ηₖ). The probability p(ηₖ) depends among other things on the spectrometer used, and can be determined during a learning phase using a set of identified spectra (Dancik et al., 1999).

The interpretation process consists in attributing to each peak from sₑₓₚ an ionic hypothesis comprising all four attributes described above. Therefore, each peak sⱼ from sᵢₙₜ will be characterized by a mass/charge ratio μ(sⱼ), an intensity ι(sⱼ), and an ionic hypothesis η(sⱼ). The number of elements in the interpreted peak list sᵢₙₜ is |sᵢₙₜ|=|sₑₓₚ|·|Δ|. This approach means that at least |Δ|−1 interpreted peaks computed from a given peak in sₑₓₚ are false.

II. Graph Construction

Let us define a spectrum graph G=(V, E) as a directed acyclic graph, with a set of vertices V={v₁, v₂, . . . , v_{|v|}} and of edges E={eᵢⱼ|i<j<|V|,vᵢ and vⱼεV}. Each vertex vᵢ is characterized by a b-mass, μ(vᵢ) and its corresponding ionic peak mass/charge ratio μ^{s}(vᵢ), an intensity ι^{s}(vᵢ), a score σ(vᵢ), an ionic hypothesis η(vᵢ), a family F(vᵢ), and a successor list succ(vᵢ), while each edge eᵢⱼεE is characterized by a pheromone trail τ(eᵢⱼ) and a label λ(eᵢⱼ).

1. Building the Vertices:

G is built from the peak list sᵢₙₜ. The first step is to transform all interpreted peaks into b-ions charged once, which represent N-terminal “ideal” fragments.

Each peak from sᵢₙₜ leads to a vertex vᵢ. Given Mₑₓₚ the experimental parent mass, with Mₑₓₚ=(M_{obs}−1)·c(M_{obs}), M_{obs} being the mass/charge ratio of the peptide parent mass, and C(M_{obs}) its charge number, we built the vertices according to algorithm 1.

Algorithm 1: Building the Vertices

```
i = 0 ;
For each sⱼ ∈ sᵢₙₜ {
        (t(η(sⱼ)) =“N − term”)
                μ(νᵢ) [symbol] c(η(sⱼ)) · μ(sⱼ)−(c(η(sⱼ))−1)−o(η(sⱼ));
        if (t(η(sⱼ)) =“C − term”)
                μ(vᵢ) [symbol] Mₑₓₚ −[c(η(sⱼ)) · μ(sⱼ)−(c(η(sⱼ))−1)−o(η(sⱼ))];
        μ^{s} (νᵢ) [symbol] μ(sⱼ);
        l^{s} (νᵢ) [symbol] normalize(l(sⱼ));
        i++;
}
```

We also create an initial vertex corresponding to the empty sequence and a final vertex corresponding to the complete sequence. Therefore, the number of vertices is equal to |sᵢₙₜ|+2.

2. Vertex Families:

For each vertex, a family F of neighbor vertices is defined. The concept of family is based on the idea that when a b-fragment is represented by several ionic peaks in sₑₓₚ, the computed b-masses μ(vᵢ) of theses peaks will be almost equal. The family building is hence based on the vertex b-mass differences, which must be lower than a specified threshold. We chose not to merge the vertices as described in (Dancik et al., 1999), because the merging process does not manage the calibration error on the peaks and depends on the parent mass accuracy, which is often quite low. Accordingly, two b-masses representing the same b-fragment and derived by ionic hypothesis of different terminal types (t(η(vᵢ))≠t(η(vᵢ))) can be quite different when compared to the b-masses obtained from ionic hypothesis of same terminal type. Such b-masses therefore cannot be merged because there are too different or, if merged can produce a new vertex with a substantially less accurate b-mass. In order to avoid this problem we do not merge the vertices, but build vertex families F(vᵢ)={vⱼ . . . v_{|F(vi)|}} containing all neighbor vertices possibly belonging to the same b-fragment. This approach allows to keep the b-mass of the vertices unchanged, and hereby fully benefit of the accuracy of the spectrometer. In addition, the algorithm used for building the families is not greedy—as is the merging algorithm proposed by Dancik, but is exact.

A vertex vⱼ is added to a family F(vᵢ) according to the following rules. First, the two vertex b-masses must be close enough. As shown in equation 1, the threshold must be adapted, depending on whether the two vertices joined in a same family are derived by ionic hypothesis of a same terminal type or of different terminal types.
|μ(vⱼ)−μ(vᵢ)|<ε Equation 1:
with ε=ε₁ if t(η(vᵢ))=tη(vⱼ)), ε=ε₂ if t(η(vᵢ))≠t(η(vⱼ)) and ε₁<ε₂

Second, the two vertex b-masses have to be issued from different ionic hypothesis (η(vᵢ) !=η(vᵢ)).

Algorithm 2: Building the Families

```
For i = 1 to |V|
        F(νᵢ) = Ø;
        test1 = TRUE;
        while (test1 ) {
                νⱼ [symbol] find the new closest vertex (νᵢ);
                if (term(νᵢ) == term(νⱼ)) ε = ε₁;
                else ε = ε₂;
                if (|νⱼ − νᵢ| < ε) {
                        test2 = TRUE;
                        For each νₖ ∈ F(νᵢ)
                                if (η(νₖ) == η(νⱼ)) : test2 = FALSE;
                        if (test2) : F(νᵢ) = F(νᵢ)∪νⱼ;
                  }
                else test1 = FALSE;
          }
  }
```

3. Scoring the Vertices:

Because the vertices are built under some assumptions, we need a value defining the credibility level of each vertex. This value is represented by a score σ(vᵢ), defined according to a non exhaustive list of criterions. Two criterions are currently taken into account, leading to a redundancy score ρ(vᵢ) and a probability score π(vᵢ).
σ(vᵢ)=ρ(v^{ᵢ}⁾\sqrt{π(vᵢ)} Equation 2:

Once the families are defined, it is possible to compute ρ(vᵢ) and π(vᵢ). The redundancy score ρ(vᵢ) must be increased according to the family size as several equivalent b-masses confirm the ionic hypothesis of vᵢ, while the probability score π(vᵢ) takes into account the occurrence probability ρ(η) of the family members:

$$Equation 3 \text{:}$$

$$π ( {v}_{i} ) = {π}_{{v}_{j} ∈ F ( {v}_{i} )} ρ ( η ( {v}_{i} ) ) \cdot {π}_{{v}_{j} ∈ F ( {v}_{i} )} ( 1 - ρ ( η ( {v}_{i} ) ) )$$

4. Connecting the Graph:

If the b-masses of two associated vertices vᵢ and vⱼ differ by the value of one or several amino acids, they can be connected by an edge eᵢⱼ. According to the number of amino-acids included in a given edge, the latter can be called a simple edge (|λ(eᵢⱼ)|=1), a double edge (|λ(eᵢⱼ)|=2), and so on. Let A={a₁,a₂, . . . , a_{|A|}} be the alphabet of the amino-acids. A contains all common amino-acids, as well as some modified amino acids, such as carboxymethylated cysteine, carbamidomethylated cysteine, or oxidated methionine. Each aᵢεA has a mass μ(aᵢ) and a label λ(aᵢ). A^{c}={a₁^{c},a₂^{c}, . . . ,a_{|A}_{^{c}}_{|}} is the set of all combinations of I to N amino acids among JAI. Because the edge number increases exponentially with the value of N, the latter is usually small (typically N=2 or N=3).

Given μ(aₙ^{c}), the sum of the masses of all amino acids in aₙ^{c}, and λ(aₙ^{c}), formed from the labels of the amino acids in aₙ^{c}, the algorithm 3 shows the computation of the edges. The vertex list must be sorted according to the b-masses values.

Algorithm 3: Connecting the Graph

```
For i = 0 to |V|
        For j = i + 1 to |V| {
                if (t(η(νᵢ)) == t(η(νⱼ))) ε = ε₁;
                else ε = ε₂;
                For n = 1 to |A^{c}| {
                        if (|μ(νⱼ)− μ(νᵢ)− μ(aₙ^{c})| < ε)
                                createEdge (eᵢⱼ, aₙ^{c});:
                  }
          }
```

III. Identification Process
1. The Peptide Database

Let D={P₁, P₂, . . . P_{|D|}} be the peptide database used for the identification. The peptides P_{c} can be obtained from the whole or a subset of nucleic or protein databases. P_{c} are characterized by three attributes. First, their sequence Q(P_{c})={a₁^{P},a₂^{P}, . . . , a_{|Q(P}_{^{c}}_{)|}^{P}} with aₙ^{P}εA. Second, their theoretical mass μ(P_{c}) (see equation 4). Third, an identification score (P_{c}).

Given the terminus mass values μ(N−term) and μ(C−term), μ(P_{c}) is obtained as follows:

$$Equation 4 \text{:}$$

$$μ ( {P}_{c} ) = μ ( N - term ) + μ ( C - term ) + {\sum }_{n = 1}^{\vert Q ( {P}_{c} ) \vert} μ ( {a}_{n}^{P} )$$

The identification process consists in comparing the peptides of D with the graph G and in correlating each peptide P_{c}εD with a score (P_{c}). Given Mₑₓₚ, the experimental parent mass of the spectrum, and r, a predetermined threshold, we have:

Algorithm 4: Identification Process

```
For c = 1 to |D|
        If (|μ(P_{c}) − Mₑₓₚ |< r )
                score(P_{c}) = compare(P_{c},G)
```

This algorithm results in a list of candidate peptides ranked by score. The following paragraph describes the compare function, which performs the comparing of a theoretical peptide with the graph.

2. Comparison Process

The comparison process between the graph G and a peptide P_{c} requires to find in G the sections best explaining P_{c}. A complete section is a path in the graph corresponding to a whole peptide sequence. We present here a possible non deterministic strategy to search, for a given P_{c}, the best complete section in G. The algorithm will be modified further in order to extract sections instead of complete paths.

Let F={f₁, f₂, . . . , f_{|F|}} be the ant population. Each ant fₖ, walking on the graph at iteration t, builds a path which includes a set of vertices L_{V}^{t}(fₖ), subset of V, such that
L_{V}^{t}(fₖ)={v₁, v₂, . . . , v_{|L}_{_{V}}_{^{t}}_{(f}_{ₖ}_{)|}}
and consequently, a set of edges, denoted L_{E}^{t}(fₖ)⊂E of size |L_{E}^{t}(fₖ)|. The quality of L_{E}^{t}(fₖ) is represented by the ant's score S^{t}(fₖ). The concatenation of the edge labels λ(eᵢⱼ), with eᵢⱼεL_{E}^{t}(fₖ), represents the sequence
L_{Q}^{t}(fₖ)={a₁^{L}, a₂^{L}, . . . , a_{|L}_{_{Q}}_{^{t}}_{(f}_{ₖ}_{)|}}, aᵢ^{L}εA^{c}
built by ant k.

Algorithm 5 is an adaptation to our problem of an ACO algorithm. First, τ(eᵢⱼ), the amount of pheromone of each edge eᵢⱼεG is initialized (with τₒ=10⁻⁶), as well as the best complete path found in the graph (L⁺) and its associated score S(L⁺). At the beginning of each iteration (tₘₐₓ is the predefined total number of iterations), the amount of pheromone that will be added at each edge, Δτ(eᵢⱼ), is initialized at 0. Then, each ant parses the graph, building its own path L_{E}^{t}(fₖ) and gets a score S^{t}(fₖ). This score is used for updating the Δτ(eᵢⱼ) for each eᵢⱼεL_{E}^{t}(fₖ). Q is a predefined constant value, chosen of a same order of magnitude as that of the optimal score. Authors have demonstrated that the value of Q has little influence on the final result (Theiler, 2001; Bonabeau et al., 1999). If the path built by the ant obtains a higher score than S(L⁺), L⁺ and S(L⁺) are updated. Finally, when all ants have parsed the graph and have added their contribution to the Δτ(eᵢⱼ), the graph is updated, ωε[0;1[ being the evaporation rate. At the end, the compare function returns the score of the best path attributed to P_{c}.

Algorithm 5: Finding the Best Path in G for a Peptide P

Initiation:

L⁺=Ø;

S(L⁺)=0;

For each edge eᵢⱼεE:τ(eᵢⱼ)=τₒ

Iterations:

For t = 1 to tₘₐₓ {

For each eᵢⱼ ∈ E:Δτ(eᵢⱼ) = 0;
For k = 1 to |F| {

(L_{V}^{t}(fₖ), L_{E}^{t}(fₖ), L_{Q}^{t}(fₖ)) = parseGraph(P_{c}, fₖ);
S^{t}(fₖ) [symbol] scoreAnt(P_{c}, fₖ, L_{V}^{t}(fₖ), L_{E}^{t}(fₖ), L_{Q}^{t}(fₖ));
$\begin{matrix}For each {e}_{ij} ε {L}_{E}^{t} ( {f}_{k} ) : Δτ ( {e}_{ij} ) = Δτ ( {e}_{ij} ) + \frac{{S}^{t} ( {f}_{k} )}{Q} ; \\ // update Δτ ( {e}_{ij} )\end{matrix}$
if (S(L⁺) < S^{t}(fₖ)) { // update best path

S(L⁺) [symbol] S^{t}(fₖ);
L⁺ [symbol] L_{E}^{t}(fₖ);

}

}
For each eᵢⱼ ∈ E:τ(eᵢⱼ) [symbol] (1 − ω) · τ(eᵢⱼ) + Δτ(eᵢⱼ); // update graph

}
return S(L⁺);

A more detailed description of the parseGraph and scoreAnt functions follows:

(a) Parsing the Graph:

The ant fₖ is first placed on the initial vertex vᵢ. It can go forward as long as the current vertex vᵢ has any successors (succ (vᵢ)≠Ø), and as long as the length of its built sequence |L_{Q}(fₖ)| is smaller than the length of the current database sequence |Q(P_{c})|. The transition rule used to go from a vertex vᵢ to a vertex vⱼ with vⱼεsucc(vᵢ) depends on three pieces of information. The first one is visibility, represented by σ(vⱼ), the score of the successor vertex. It can be considered as a local parameter. The second piece of information corresponds to the memory of the learning previously done by the ant population. It is a global parameter, representing the amount of pheromone laid on the edge eᵢⱼ, τ(eᵢⱼ). Finally, the third piece of information is the sequence of the current database peptide P_{c}. Indeed, if the label of the next edge eᵢⱼ matches the next amino acid in the sequence Q(P_{c}), the transition probability is multiplied by a predefined constant value dependent upon the edge label length.

Given αand β, two adjustable parameters controlling the relative weight of the learning and the visibility, pₜ^{fx}(eᵢⱼ), the probability for ant fₖ to take the edge eᵢⱼ at iteration t, pₜ^{fx}(eᵢ) the set of these probabilities for all succ(vᵢ), and Q(P_{c})={a₁^{P}, a₂^{P}, . . . , a_{|Q(P}_{_{c}}_{)|}^{P}}, the current peptide sequence:

$$Algorithm 6 \text{:} Parsing G with ant {f}_{k}$$

$$i = 1$$

$${L}_{E}^{t} ( {f}_{k} ) = ∅ ; {L}_{V}^{t} ( {f}_{k} ) = ∅ ; {L}_{Q}^{t} ( {f}_{k} ) = ∅ ;$$

$$while ( succ ( {v}_{i} ) = ∅ ) and ( \vert {L}_{Q}^{t} ( {f}_{k} ) \vert < \vert Q ( {P}_{c} ) \vert ) \text{\{}$$

$$for each {v}_{j} ∈ succ ( {v}_{i} ) \text{\{}$$

$${p}_{t}^{{f}_{k}} ( {e}_{ij} ) = \frac{{τ ( {e}_{ij} )}^{α} \cdot {σ ( {e}_{ij} )}^{β}}{{\sum }_{∀ succ ( {v}_{i} )} ( {τ ( {e}_{ij} )}^{α} \cdot {σ ( {e}_{ij} )}^{β} )} ;$$

$$if ( match ( {a}_{\vert {L}_{Q}^{t} ( {f}_{k} ) \vert + 1}^{P} , … , {a}_{\vert {L}_{Q}^{t} ( {f}_{k} ) \vert + \vert λ ( {e}_{ij} ) \vert}^{P} , λ ( {e}_{ij} ) ) ) : {p}_{t}^{{f}_{x}} ( {e}_{ij} ) = {p}_{t}^{{f}_{x}} ( {e}_{ij} ) \cdot {c}_{\vert λ ( {e}_{ij} ) \vert} ;$$

$$\text{//} here , we compare all permutations in λ ( {e}_{ij} )$$

$$with the amino acids {a}_{\vert {L}_{Q}^{t} ( {f}_{k} ) \vert + 1}^{P} , … , {a}_{\vert {L}_{Q}^{t} ( {f}_{k} ) \vert + \vert λ ( {e}_{ij} ) \vert}^{P}$$

$$add ( {p}_{t}^{{f}_{x}} ( {e}_{i} ) , {p}_{t}^{{f}_{x}} ( {e}_{ij} ) ) ;$$

$$\{ \\ normalize ( {p}_{t}^{{f}_{x}} ( {e}_{i} ) ) ; \\ {e}_{ij} = chooseEdge ( {p}_{t}^{{f}_{x}} ( {e}_{i} ) ) ; \\ add ( {L}_{V}^{t} ( {f}_{k} ) , {v}_{j} ) ; \\ add ( {L}_{E}^{t} ( {f}_{k} ) , {e}_{ij} ) ; \\ add ( {L}_{Q}^{t} ( {f}_{k} ) , λ ( {e}_{ij} ) ) ; \\ i ← j ; \\ \text{\}}$$

(b) Scoring the Ants

At the end of each iteration t, one must evaluate the similarity between the current peptide P_{c} and the different paths used by the ants. Each ant gets a final score S^{t}(fₖ) depending on its path L_{E}^{t}(fₖ). The goal is to include in S^{t}(fₖ) all possibly relevant information from different sources (see equation 5). For example, in order to take into account information coming from Sᵢₙₜ we can use the intensity of the peaks, stored in ι^{s}(vᵢ), vᵢεL_{V}^{t}(fₖ), and compute an intensity score intS. From the ionic hypothesis set, we can build a relevancy score relS, expressing the relevancy of the vertices parsed by fₖ. The current peptide sequence can be used in a covS score that would express the similarity between the peptide sequence Q(P_{c}) and the sequence L_{Q}^{t}(fₖ) built by the ant. The quality of the correlation between the b-masses of the used vertices and the theoretical masses expected from Q(P_{c}) can also be taken into account as a regression score called regS. Still other information can be added, such as rules resulting from the expertise of biologists used to studying MS/MS data.
S^{t}(fₖ)=f(intS,relS,covS,regS, . . . ); Equation 5:

The next sections show implementation examples of the sub-scores intS, relS, covS and regS used in our current algorithm.

The coverage score recS represents the sequence similarity between the current peptide P_{c} and the sequence built by an ant fₖ. It is computed with an alignment function as for example a Smith and Waterman algorithm. Given Q(P_{c}) and L_{Q}^{t}(fₖ):

Algorithm 7: Coverage Score
recS=align(Q(P_{c}), L_{Q}^{t}(fₖ));

The relevancy score is the mean of the used vertices score. It is computed as shown in equation 6.

$$Equation 6 \text{:}$$

$$relS = \frac{{\sum }_{{v}_{i} ∈ {L}_{V}^{t} ( {f}_{k} )} σ ( {v}_{i} )}{\vert {L}_{V}^{t} ( {f}_{k} ) \vert}$$

Similarly, the intensity score is computed as follows:

$$Equation 7 \text{:}$$

$$intS = \frac{{\sum }_{{v}_{i} ∈ {L}_{V}^{t} ( {f}_{k} )} {t}^{s} ( {v}_{i} )}{\vert {L}_{V}^{t} ( {f}_{k} ) \vert}$$

The regression score measures the global correspondence between the experimental masses μ^{s}(vᵢ) of the vertices included in the ant's path and the corresponding theoretical masses R(P_{c})={r₁, r₂, . . . , r_{|R(P}_{_{c}}_{)|}} computed from the current database peptide sequence Q(P_{c}) (Gras et al., 2000). The relation between these masses is first plotted on a graph, with the experimental masses as abscissa and the theoretical masses as ordinate, and the set of points allows to calculate a linear regression. The mean of the deviation between the points and the linear regression represents the regression score regS.

Given y=ax+b, the linear regression, μ^{s}(vᵢ)εL_{V}^{t}(fₖ) the experimental masses and their corresponding theoretical masses rᵢεR(P_{c}):

Algorithm 8: Computation of regS

For each μ^{s}(vᵢ) ∈ L_{V}^{t} (fₖ) {

| | | |
|---|---|---|
| add (R, μ^{s}(vᵢ), Q(P_{c})); | // | compute the corresponding |
| | | theoretical mass rᵢ and add |
| linearReg(a,b,R,L_{V}^{t} (fₖ)); | // | it to R this function makes |
| | | the regression |

$regS = \frac{{\sum }_{i = 0}^{\vert {L}_{V}^{t} ( {f}_{k} ) \vert} {( a \cdot {r}_{i} - {μ}^{s} ( {v}_{i} ) + b )}^{2}}{\vert {L}_{V}^{t} ( {f}_{k} ) \vert}$
}

### EXPERIMENTAL EXAMPLE

A preliminary implementation of our algorithm has been tested on a training set of MS/MS spectra (only complete paths, no unknown modifications). 92.1% of 101 spectra were well identified. Here are some result examples.

| MSMS file | DSNNLXLHFNPR.dta |
|---|---|
| Peaks used/tot | 56/935 |
| Parent_mass (M/H+)/charge | 1485.63/2 |
| Vertices | 170 |
| Edges (simple/double) | 482/4345 |
| Ants nb/Iter nb: | 101/5 |

| # | s_n* | fin_s** | access | id | sequence_dtb/sequence_graph |
|---|---|---|---|---|---|
| 1. | 0 | 1.396 | P09382 | LEG1_HUMAN | DSNNLCLHFNPR*** |
| | | | | | sdNNLXLHFNPR**** |
| 2. | 0 | 0.312 | Q05586 | NMZ1_HUMAN | FANYSIMNLQNR |
| | | | | | ewNIsinmLPNR |
| 3. | 0 | 0.252 | P09848 | LPH_HUMAN | DPSNQEDVEAARR |
| | | | | | rxLNQEvdaePR |

*s_n = start node
**fin_s = final score
***theoretical sequence read in the database
****sequence parsed in the graph (uppercase = simple edge, lower case = double edge)

| MSMS file | EFTNVYIK.dta |
|---|---|
| Peaks used/tot | 40/260 |
| Parent_mass (M/H+)/charge | 1012.51/2 |
| Vertices | 122 |
| Edges (simple/double) | 349/3153 |
| Ants nb/Iter nb: | 74/5 |

| | | | | | sequence_dtb/ |
|---|---|---|---|---|---|
| # | s_n* | fin_s** | access | id | sequence_graph |
| 1. | 0 | 1.970 | Q13310 | PAB4_HUMAN | EFTNVYIK |
| | | | | | EFTNVYIK |
| | 0 | 1.970 | Q15097 | PAB2_HUMAN | EFTNVYIK |
| | | | | | EFTNVYIK |
| | 0 | 1.970 | P11940 | PAB1_HUMAN | EFTNVYIK |
| | | | | | EFTNVYIK |
| 2. | 0 | 1.079 | P42694 | Y054_HUMAN | QDYEMALK |
| | | | | | ADeyaoLK |
| 3. | 0 | 0.677 | P46821 | MAPB_HUMAN | LKHLDFLK |
| | | | | | LKlhdfLK |

| MSMS file | EQIVPKPEEEVAQK.dta |
|---|---|
| Peaks used/tot | 64/317 |
| Parent_mass (M/H+)/charge | 1622.83/3 |
| Vertices | 194 |
| Edges (simple/double) | 579/4566 |
| Ants nb/Iter nb: | 120/5 |

| # | s_n* | fin_s** | access | id | sequence_dtb/sequence_graph |
|---|---|---|---|---|---|
| 1. | 0 | 1.374 | P18621 | RL17_HUMAN | EQIVPKPEEEVAQK |
| | | | | | qeviPKPEEEVAQK |
| 2. | 0 | 0.396 | P36383 | CXA7_HUMAN | LLEEIHNHSTFVGK |
| | | | | | LLEEvkCHSvzVG |
| 3. | 0 | 0.394 | P16991 | YB1_HUMAN | RPENPKPQDGKETK |
| | | | | | RPtdPKPQvxgiQK |

1. A peptide identification method comprising the following steps:
(a) performing tandem mass spectrometry on a sample containing one or more protein or peptide;
(b) reducing the resulting spectrum to a peak list;
(c) listing possible interpretations for said peak list into an interpreted peak list, taking into account physico-chemical knowledge;
(d) structuring said interpreted peak list into a structured representation taking into account biological knowledge wherein said structuring comprises preserving at least the mass to charge ratio of the peaks obtained in step (b), the mass to charge ratio of the peptide or protein, the charge of the peptide or protein, and the intensity of the peaks obtained in step (b);
(e) matching said structured representation with a biological sequence database prior to any reduction of the structured information into one or a limited number of amino acid sequences; and
(f) determining the best peptide match or matches within said database.

2. The method of claim 1, and further comprising a step (g) comprising using the peptide matching information of step (f) for identification of the corresnonding protein or proteins in the protein database.

3. The method of claim 1 wherein the structured representation of step (d) comprises a graph wherein vertices of the graph represent individual elements of the interpreted peak list, translated into potential b-ion type peptide fragments and edges link vertices representing said b-ion type peptide fragments whose molecular weights differ by a value equivalent to the molecular weight of one or more amino acids.

4. The method of anyone of claim 1 wherein the matching of step (e) comprises successively parsing the structured representation of step (d) according to each database sequence, each parsing leading to a score correlating each database sequence to the structured representation.

5. The method of claim 4 wherein the parsing is performed by a Swarm Intelligence Algorithm.

6. The method of claim 5 wherein the Swarm Intelligence algorithm is an Ant Colony Optimization algorithm.

7. The method of anyone of claim 3 wherein non-linked relevant sets of successive edges are combined together according to a modification hypothesis.

8. A computer-readable medium comprising instructions for causing a computer linked to one or several mass spectrometers and to one or more biological sequence databases to perform the steps of the method of anyone of claim 1.

9. A system comprising a computer linked to one or more mass spectrometers and to one or more biological sequence databases, said computer comprising a program for performing the steps of the method of anyone of claim 1.

10. A peptide identification method comprising the following steps:
(a) performing tandem mass spectrometry on a sample containing one or more protein or peptide;
(b) reducing the resulting spectrum to a peak list;
(c) listing possible interpretations for said peak list into an interpreted peak list, taking into account physico-chemical knowledge;
(d) structuring said interpreted peak list into a structured representation taking into account biological knowledge wherein said structuring comprises preserving at least the mass to charge ratio of the peaks obtained in step (b), the mass to charge ratio of the peptide or protein, the charge of the peptide or protein, and the intensity of the peaks obtained in step (b), and wherein said structured representation comprises a graph wherein vertices of the graph represent individual elements of the interpreted peak list, translated into potential b-ion type peptide fragments and edges link vertices representing said b-ion type peptide fragments whose molecular weights differ by a value equivalent to the molecular weight of one or more amino acids;
(e) matching said structured representation with a biological sequence database prior to any reduction of the structured information into one or a limited number of amino acid sequences;
(f) determining the best peptide match or matches within said database; and
(g) using the peptide matching information of step (f) for identification of the corresponding protein or proteins in the protein database.

11. The method of anyone of claim 10 wherein the matching of step (e) comprises successively parsing the structured representation of step (d) according to each database sequence, each parsing leading to a score correlating each database sequence to the structured representation.

12. The method of claim 10 wherein the parsing is performed by a Swarm Intelligence Algorithm.

13. The method of claim 10 wherein the Swarm Intelligence algorithm is an Ant Colony Optimization algorithm.

14. The method of anyone of claim 10 wherein non-linked relevant sets of successive edges are combined together according to a modification hypothesis.

15. A computer-readable medium comprising instructions for causing a computer linked to one or several mass spectrometers and to one or more biological sequence databases to perform the steps of the method of anyone of claim 10.

16. A system comprising a computer linked to one or more mass spectrometers and to one or more biological sequence databases, said computer comprising a program for performing the steps of the method of anyone of claim 10.

17. A peptide identification method comprising the following steps:
(a) performing tandem mass spectrometry on a sample containing one or more protein or peptide;
(b) reducing the resulting spectrum to a peak list;
(c) listing possible interpretations for said peak list into an interpreted peak list, taking into account physico-chemical knowledge;
(d) structuring said interpreted peak list into a structured representation taking into account biological knowledge wherein said structuring comprises preserving at least the mass to charge ratio of the peaks obtained in step (b), the mass to charge ratio of the peptide or protein, the charge of the peptide or protein, and the intensity of the peaks obtained in step (b);
(e) matching said structured representation with a biological sequence database prior to any reduction of the structured information into one or a limited number of amino acid sequences, wherein the matching comprises successively parsing the structured representation of step (d) according to each database sequence, each parsing leading to a score correlating each database sequence to the structured representation;
(f) determining the best peptide match or matches within said database; and
(g) using the peptide matching information of step (f) for identification of the corresponding protein or proteins in the protein database.

18. The method of claim 17 wherein the parsing is performed by a Swarm Intelligence Algorithm.

19. The method of claim 17 wherein the Swarm Intelligence algorithm is an Ant Colony Optimization algorithm.

20. The method of anyone of claim 17 wherein non-linked relevant sets of successive edges are combined together according to a modification hypothesis.
